(19) United States
(12) Patent Application Publication
(10) Pub. No.: US 2005/0119562 A1
(43) Pub. Date: Jun. 2, 2005

(54) Fibrous marker formed of synthetic polymer strands

(75) Inventors: Michael L. Jones, San Clemente, CA (US); Paul Lubock, Laguna Niguel, CA (US); Lloyd H. Malchow, San Juan Capistrano, CA (US)

Correspondence Address: Edward J. Lynch;DUANE MORRIS LLP, One Market, Spear Tower, Suite 2000, San Francisco, CA, 94105, US

(73) Assignee: SenoRx, Inc.

(21) Appl. No.: 10/976,138

(22) Filed: Oct. 27, 2004

Related U.S. Application Data
(63) Continuation-in-part of application No. 10/444,770, filed on May 23, 2003.

(51) Int. Cl. A61B 5/05

(52) U.S. Cl. 600/426

(57) ABSTRACT

The invention is directed to an intracorporeal fibrous marker formed of a fibrous pad or mat with strands of synthetic polymeric material such as polyglycolic acid and delivery devices and methods of using such devices. The fibrous pad or mat has a bulk density of at least 10, preferably about 30 to about 100 mg/cc and typically about 40 mg/cc. The fibrous marker has an effective in vivo lifespan of at least three weeks.

### RELATED APPLICATIONS

This application is a continuation-in-part of application Ser. No. 10/444,770, filed on May 23, 2003 which is incorporated herein by reference in its entirety and from which priority is claimed.

### FIELD OF THE INVENTION

The invention is generally directed to remotely detectable, intracorporeal markers and devices and methods for the delivery of such markers to a desired location within a patient's body.

### BACKGROUND OF THE INVENTION

In diagnosing and treating certain medical conditions, it is often desirable to mark a suspicious body site for the subsequent taking of a biopsy specimen, for delivery of medicine, radiation, or other treatment, for the relocation of a site from which a biopsy specimen was taken, or at which some other procedure was performed. As is known, obtaining a tissue sample by biopsy and the subsequent examination are typically employed in the diagnosis of cancers and other malignant tumors, or to confirm that a suspected lesion or tumor is not malignant. The information obtained from these diagnostic tests and/or examinations is frequently used to devise a therapeutic plan for the appropriate surgical procedure or other course of treatment.

In many instances, the suspicious tissue to be sampled is located in a subcutaneous site, such as inside a human breast. To minimize surgical intrusion into a patient's body, it is often desirable to insert a small instrument, such as a biopsy needle, into the body for extracting the biopsy specimen while imaging the procedure using fluoroscopy, ultrasonic imaging, x-rays, magnetic resonance imaging (MRI) or any other suitable form of imaging technique or palpation. Examination of tissue samples taken by biopsy is of particular significance in the diagnosis and treatment of breast cancer. In the ensuing discussion, the biopsy and treatment site described will generally be the human breast, although the invention is suitable for marking biopsy sites in other parts of the human and other mammalian body as well.

Periodic physical examination of the breasts and mammography are important for early detection of potentially cancerous lesions. In mammography, the breast is compressed between two plates while specialized x-ray images are taken. If an abnormal mass in the breast is found by physical examination or mammography, ultrasound may be used to determine whether the mass is a solid tumor or a fluid-filled cyst. Solid masses are usually subjected to some type of tissue biopsy to determine if the mass is cancerous.

If a solid mass or lesion is large enough to be palpable, a tissue specimen can be removed from the mass by a variety of techniques, including but not limited to open surgical biopsy, a technique known as Fine Needle Aspiration Biopsy (FNAB) and instruments characterized as “vacuum assisted large core biopsy devices”.

If a solid mass of the breast is small and non-palpable (e.g., the type typically discovered through mammography), a vacuum assisted large core biopsy procedure is usually used. In performing a stereotactic biopsy of a breast, the patient lies on a special biopsy table with her breast compressed between the plates of a mammography apparatus and two separate x-rays or digital video views are taken from two different points of view. A computer calculates the exact position of the lesion as well as depth of the lesion within the breast. Thereafter, a mechanical stereotactic apparatus is programmed with the coordinates and depth information calculated by the computer, and such apparatus is used to precisely advance the biopsy needle into the small lesion. The stereotactic technique may be used to obtain histologic specimens. Usually at least five separate biopsy specimens are obtained from locations around the small lesion as well as one from the center of the lesion.

The available treatment options for cancerous lesions of the breast include various degrees of mastectomy or lumpectomy, radiation therapy, chemotherapy and combinations of these treatments. However, radiographically visible tissue features, originally observed in a mammogram, may be removed, altered or obscured by the biopsy procedure, and may heal or otherwise become altered following the biopsy. In order for the surgeon or radiation oncologist to direct surgical or radiation treatment to the precise location of the breast lesion several days or weeks after the biopsy procedure was performed, it is desirable that a biopsy site marker be placed in the patient's body to serve as a landmark for subsequent location of the lesion site. A biopsy site marker may be a permanent marker (e.g., a metal marker visible under x-ray examination), or a temporary marker (e.g., a bioresorbable marker detectable with ultrasound). While current radiographic type markers may persist at the biopsy site, an additional mammography generally must be performed at the time of follow up treatment or surgery in order to locate the site of the previous surgery or biopsy. In addition, once the site of the previous procedure is located using mammography, the site must usually be marked with a location wire which has a hook on the end which is advanced into site of the previous procedure. The hook is meant to fix the tip of the location wire with respect to the site of the previous procedure so that the patient can then be removed from the confinement of the mammography apparatus and the follow-up procedure performed. However, as the patient is removed from the mammography apparatus, or otherwise transported the position of the location wire can change or shift in relation to the site of the previous procedure. This, in turn, can result in follow-up treatments being misdirected to an undesired portion of the patient's tissue.

As an alternative or adjunct to radiographic imaging, ultrasonic imaging (herein abbreviated as “USI”) or visualization techniques can be used to image the tissue of interest at the site of interest during a surgical or biopsy procedure or follow-up procedure. USI is capable of providing precise location and imaging of suspicious tissue, surrounding tissue and biopsy instruments within the patient's body during a procedure. Such imaging facilitates accurate and controllable removal or sampling of the suspicious tissue so as to minimize trauma to surrounding healthy tissue.

For example, during a breast biopsy procedure, the biopsy device is often imaged with USI while the device is being inserted into the patient's breast and activated to remove a sample of suspicious breast tissue. As USI is often used to image tissue during follow-up treatment, it may be desirable to have a marker, similar to, the radiographic markers discussed above, which can be placed in a patient's body at the site of a surgical procedure and which are visible using USI. Such a marker enables a follow-up procedure to be performed without the need for traditional radiographic mammography imaging which, as discussed above, can be subject to inaccuracies as a result of shifting of the location wire as well as being tedious and uncomfortable for the patient.

Placement of a marker or multiple markers at a location within a patient's body requires delivery devices capable of holding markers within the device until the device is properly situated within a breast or other body location. Accordingly, devices and methods for retaining markers within a marker delivery device while allowing their expulsion from the devices at desired intracorporeal locations are desired.

### SUMMARY OF THE INVENTION

The invention is generally directed to a remotely imageable, fibrous marker suitable for deployment at a site within a patient's body, particularly a biopsy site such as in a patient's breast. The fibrous marker is formed of a synthetic polymer strands and is imageable by ultrasound for at least three weeks, preferably about one month or more at the intracorporeal site. The fibrous marker is preferably in the form of a fibrous pad or mat, e.g. felt, formed of strands of synthetic polymeric material and has a bulk density greater than 10 mg/cc, preferably about 30 to about 100 mg/cc. Preferably the synthetic polymer strands are hydrophobic. Suitable commercially available felt matting has a bulk density of about 40 mg/cc. Preferably synthetic polymeric materials are predominantly polyglycolic acid (PGA), i.e. at least 50% (by weight) and preferably is about 85% (by weight) to 100% (by weight) PGA. Other synthetic polymeric materials include polylactic acid, polycaprolactone and copolymers thereof and therewith.

The fibrous marker preferably includes a radiopaque element, such as a metallic ring or clip, for long term identification of the intracorporeal site. Preferably, the radiopaque element is formed of non-magnetic material to avoid interference with magnetic resonance imaging (MRI). Suitable non-magnetic materials include titanium, platinum, gold, iridium, tantalum, tungsten, silver, rhodium, non-magnetic stainless steel (316) and the like. The radiopaque element should have a non-natural shape so that it is readily recognized when remotely imaged. The radiopaque element should have a maximum dimension of about 0.5 to about 5 mm, preferably about 1 to about 3 mm to ensure identification, particularly with MRI.

The fibrous body of the marker is formed into a an elongated member suitable for delivery by rolling or folding a fibrous mat or pad, and binding the rolled or folded mat or pad in a compressed condition to provide sufficient column strength to facilitate introduction into and discharge of the compressed and rolled body from a tubular delivery device. Suitable binding agents for holding the fibrous marker in a compressed condition are water soluble polymers such as polyvinyl alcohol, polyethylene glycol and polyvinyl pyrollidone. One or more radiographically detectable, and preferably non-magnetic marker elements are provided with the fibrous marker. The radiopaque marker element is preferably centrally located on the elongated body to ensure that the radiographically detectable element is disposed at a more or less central location within the target site rather than at a site margin. In one embodiment the radiopaque element is a ring which encircles a mid-point of the rolled or folded fibrous body.

The fibrous marker embodying features of the invention can be readily delivered to the desired location by suitable delivery systems. The marker delivery system generally has an elongated cannula or syringe-like body with proximal and distal ports and an inner lumen extending between the ports. The fibrous marker is slidably disposed within the inner lumen of the delivery cannula and a plunger slidably disposed within the inner lumen of the delivery cannula proximal to the marker. The plunger is movable from an initial position proximal to the fibrous marker within the tube, to a delivery position close to the discharge opening in the distal end of the cannula to push the fibrous marker out of the discharge opening into the target tissue site.

Upon being discharged into the intracorporeal target site, body fluid at the site infiltrates the fibrous marker and the marker at least partially fills the site to enable short term detection (at least three weeks, preferably at least four weeks but less than a year) by remote USI and preferably long term detection by remote mammographic imaging or MRI identification. When the compressed fibrous marker body is infiltrated with body fluid, e.g. blood, within the biopsy cavity or other intracorporeal site, the binding agent in the marker body is dissolved so the fibrous body can expand within the intracorporeal site. While the marker body takes up water, the individual strands which form the marker do not swell significantly (less than 5%) on contact with body fluid. The expanded fibrous marker positions the radiopaque marker element within the interior of the target cavity.

The cannula of the marker delivery device may be configured to fit within the guide cannula of a biopsy device, such as a Mammotome® (sold by Johnson & Johnson), the SenoCor 360™ biopsy device sold by SenoRx (the present assignee), the EnCor,™ biopsy device sold by SenoRx and or a coaxial needle guide. The delivery cannula can also be configured to fit into the proximal end of a tubular cutting element such as found in the EnCor™ biopsy system sold by SenoRx which is the subject of co-pending application Ser. No. 10/911,206, filed on Aug. 3, 2004.

One suitable delivery system suitable for delivery through a tubular cutter (e.g. as with the Oncore™system) is a syringe-type delivery system described in co-pending application Ser. No. 10/911,206, filed on Aug. 3, 2004) having a tubular shaft with a flared guide on or integral with the distal tip to facilitate engagement with the proximal end of the tubular cutter. Another syringe-type delivery system has a plugged distal tip to prevent body fluids from engaging one or more markers which may be in the tubular shaft of the delivery system. Such fluid infusions can retard or restrict discharging the fibrous marker and other markers which may be within the inner lumen of the delivery cannula. Delivery systems with plugged tips are described in co-pending applications Ser. No. 10/444,770, filed on May 23, 2003 and Ser. No. 10/753,277, filed on Dec. 23, 2003, which are incorporated herein in their entireties. The plugged tip type delivery systems can have a side opening for marker deployment or a plugged needle-type distal tip both of which are disclosed in the above application Ser. No. 10/753,694.

A variety of therapeutic or diagnostic agents may be incorporated into the fibrous marker. Incorporated agents can include for example, hemostatic agents to form thrombus at the intracorporeal site, anesthetic agents to control pain, chemotherapeutic agents for treating residual neoplastic tissue or coloring agents to facilitate subsequent visual location of the site. Antibiotics, antifungal agents and antiviral agents may also be incorporated into the fibrous marker.

Upon delivery to the intracorporeal site, the fibrous marker unrolls and expands to facilitate identification and localization. The marker is easily identifiable from surrounding tissue at the site by ultrasonic imaging (USI).

The fibrous markers embodying features of the present invention provide several advantages. The synthetic polymeric strands are preferably hydrophobic which eases the difficulty in manufacturing the markers because they do not react with surrounding moisture. Moreover, the fibrous marker material is stabilized quickly in the intracavity clot which forms at the biopsy site and can be readily identified from surrounding tissue of the cavity by less skilled radiologists or surgeons.

These and other advantages of the invention will become more apparent from the following detailed description of embodiments when taken in conjunction with the accompanying exemplary drawings.

### BRIEF DESCRIPTION OF THE DRAWINGS

FIG. 1 is an elevational view of a fibrous marker embodying features of the invention.

FIG. 2 is a perspective end view of the fibrous marker shown in FIG. 1 illustrating the rolled structure of the marker.

FIG. 3A is a partly cut-away perspective view of a marker delivery assembly for the fibrous marker shown in FIG. 1.

FIG. 3B is a transverse cross-sectional view of the marker delivery assembly of FIG. 3A taken at line 3B-3B.

FIG. 3C is a transverse cross-sectional view of the marker delivery assembly of FIG. 3A taken at line 3C-3C.

FIG. 4 is a longitudinal cross-sectional view which illustrates a partially cut away, perspective view of a human breast from which a biopsy specimen has been removed, showing a fibrous marker being delivered to the biopsy site with the marker delivery assembly shown in FIG. 3A.

FIG. 5 is a partial cut-away view of a human female breast shown in FIG. 7 with the fibrous marker expanded in the cavity of the biopsy site with the delivery device removed.

FIG. 6 is an elevational view of an alternative marker delivery device with a flared guide on the distal end of the shaft thereof.

FIG. 7 is an enlarged partial view of the shaft of the device of FIG. 4 shown partially in section.

FIG. 8 is a longitudinal cross-sectional view which illustrates mounting the distal end of the marker delivery device shown in FIG. 4 onto the proximal end of a cutting member of a biopsy device (not shown).

FIG. 9 is a longitudinal cross-sectional view of the distal end of a biopsy device illustrating alignment of the discharge aperture of the marker delivery device shown in FIG. 6 and the tissue accessing aperture of the cannula of the biopsy device.

FIG. 10 is a longitudinal cross-sectional view of the distal portion of an alternative marker delivery device embodying features of the invention with a sharp, tissue penetrating distal tip.

### DETAILED DESCRIPTION OF EMBODIMENTS OF THE INVENTION

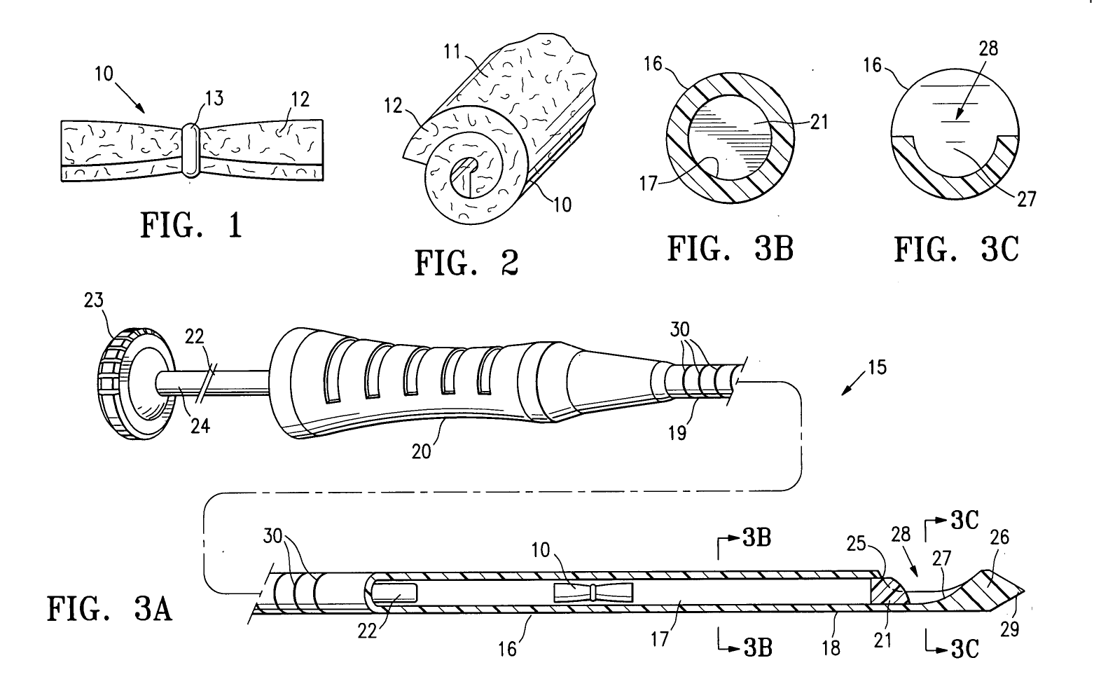

FIGS. 1 and 2 illustrate a fibrous marker 10 embodying features of the invention. The fibrous marker 10 is a rolled body 11 formed of fibers or strands 12 with a radiopaque non-magnetic ring or wireform 13 encircling at least part of the central portion of the rolled body. The fiber or strands 12 are formed of bioabsorbable synthetic polymeric material that is essentially hydrophobic and has an effective in-vivo life-span of at least three weeks, preferably at least four weeks. The fibrous marker body 11 is a rolled (or folded) and preferably compressed fibrous mat with binding material incorporated into the fibrous body to maintain the compressed condition. The rolled fibrous body 11 may be formed from a felt (as shown) or woven material. Preferably, the binding agent is a water soluble polymer such as polyethylene glycol which is incorporated into the fibrous body 11 and the body compressed to reduce the profile of the body and facilitate sliding the rolled body through a lumen of a marker delivery system to the desired site within the patient's body.

One suitable marker delivery system fibrous marker delivery system 15 is depicted in FIGS. 3A-3C which includes a delivery tube or cannula 16 with an inner lumen 17, a distal portion 18, and a proximal portion 19 with a handle 20. A releasable distal plug 21 and the fibrous marker 10 are shown disposed within the inner lumen 17. A plunger 22 is slidably disposed within the inner lumen 17 and is provided with a head 23 on the proximal end 24 configured to allow an operator to press the plunger further into the inner lumen and push both the releasable plug 21 and fibrous marker 10 out of the discharge port or opening 25 in the distal end 26 of delivery cannula 16. Cannula handle 20 allows an operator to hold the cannula steady while pressing plunger 22 to discharge the releasable plug 21 and fibrous marker 10.

Releasable plug 21 may substantially fill the discharge opening 25, as shown in FIG. 3A, or may occupy or block only a portion of the discharge opening. The exposed face of plug 21 is preferably provided with an inclined configuration. Releasable plug 21 is configured to be tight enough, e.g. press fit, in the inner lumen 17 to prevent its inadvertent release which might allow premature discharge of marker 10 from delivery cannula 16, but the plug must be easily released when the plunger 22 is pressed deeper into the inner lumen 17 of the delivery cannula 16. An adhesive or mechanical element(s) may be used to hold the releasable plug 21 in a position within the inner lumen 17 to occlude the discharge opening 25. Suitable adhesives include polyurethane or polyacrylic based adhesives, polyhydroxymethacrylate base adhesives, fibrin glue (e.g., Tisseal™), collagen adhesive, or mixtures thereof. Suitable mechanical means for securing the releasable plug 21 are described in co-pending application Ser. No. 10/174,401 which is incorporated herein by reference. The distal end 26 of the delivery cannula 16 is provided with a ramp 27 which guides the discharged plug 21 and marker 10 out of the side-port 28 into the target site. The distal tip 29 may be tapered for delivery through a guide tube (not shown).

The delivery cannula 16 may be provided with markings 30 which serve as visual landmarks to aid an operator in accurately placing the distal portion 18 of the cannula 16 in a desired location within a patient's body for discharging the marker 10.

The exterior of the delivery cannula 16 is preferably configured to fit within a guide cannula sized to accept a Mammotome®, Tru-Cut®, SenoCor® or EnCor™ biopsy device. Typically, plug 21 and marker 10 will have diameters determined by the size of the inner lumen 17 and typically will be about 0.02 inch (0.5 mm) to about 0.5 inch (12 mm), preferably about 0.04 inch (1 mm) to about 0.3 inch (8 mm). Plug 21 may have slightly larger transverse dimensions to provide a tight fit.

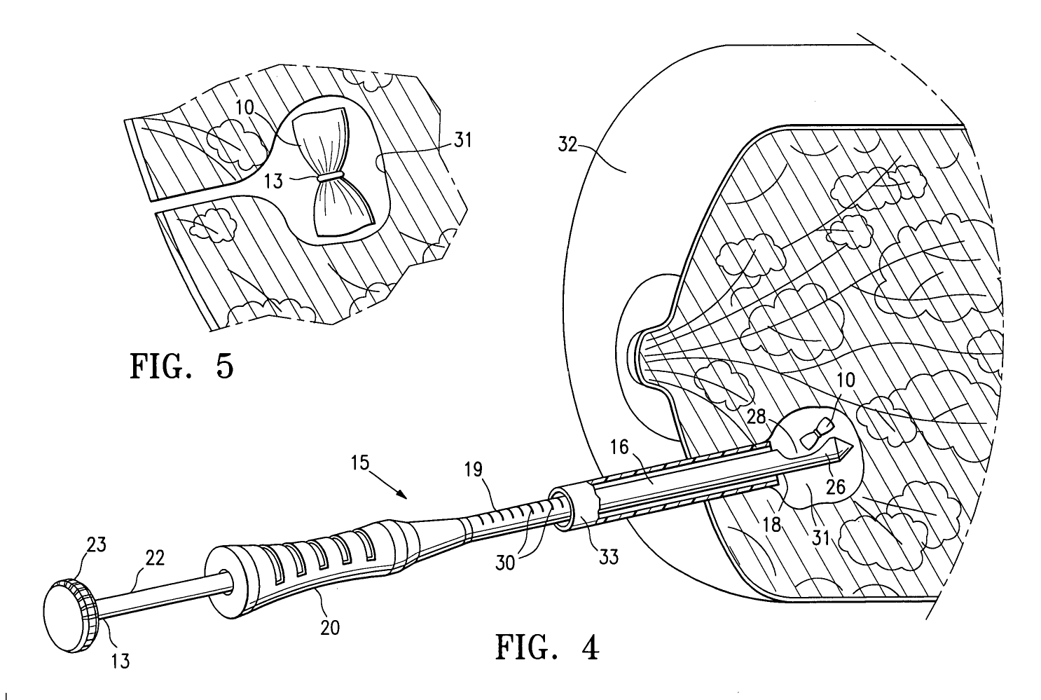

FIG. 4 schematically illustrates the delivery of fibrous marker 10 to a cavity 31 such as a biopsy site in a patient's body. The distal portion of the cannula 16 marker delivery system 15 is shown inserted into a breast 32 through a guide cannula 33 until the distal end is disposed in the cavity 31 where a tissue specimen has been removed. While an operator holds the system 10 by the handle 25 of the delivery tube 21, the plunger 20 is pressed further into the bore 12 of delivery cannula 11 to discharge the releasable plug 16 and marker 10 into the cavity 31. FIG. 5 schematically illustrates the marker 10 within the cavity 31 after deployment. When the marker 10 contacts body fluid within the cavity 31, the binding agent is dissolved and the fibrous body 11 of the marker 10 expands to further fill the cavity 31.

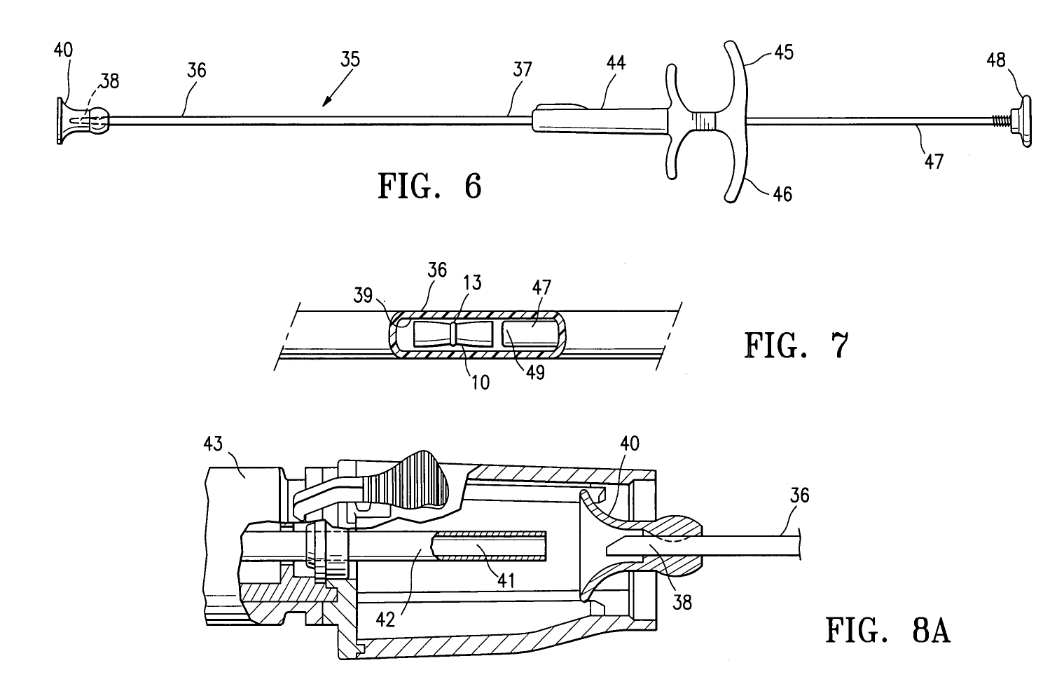

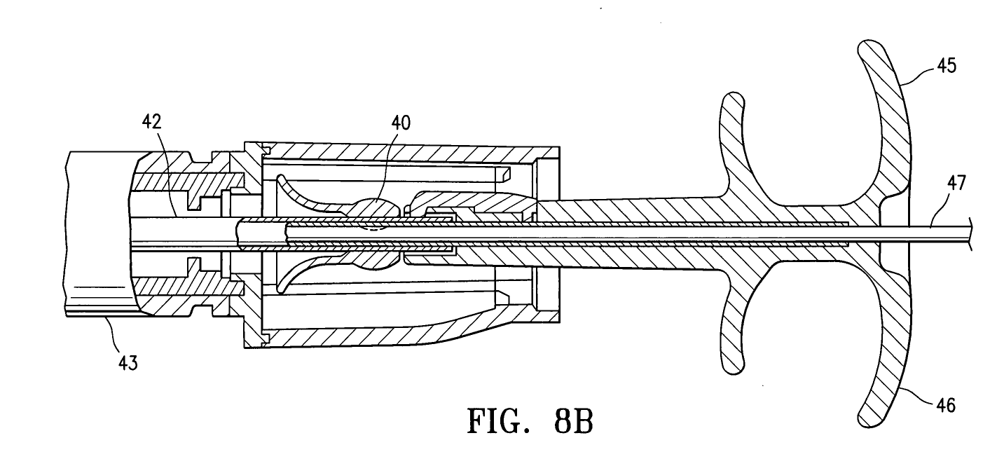

Another suitable marker delivery system 35 is shown in FIGS. 6-9 which had an elongated cannula or shaft 36 with a proximal end 37 and distal end 38, an inner lumen 39 configured to slidably receive the fibrous marker 10. A flared guide 40 is disposed on the distal end 38 to guide the distal end of the cannula 36 into the proximal end 41 of a tubular cutter 42 of a biopsy device 43 (shown in part). The proximal end 37 of cannula 36 is secured to hub 44 which has finger grips 45 and 46 for holding and manipulating the system 35. A plunger 47 is slidably disposed within the inner lumen 39 of the cannula 36 and is provided with an enlarged head 48 to facilitate the application of pressure by the operator's finger to drive the plunger further into the inner lumen 39, drive the fibrous marker 10 through the lumen and to discharge the fibrous marker from the aperture 50 in the distal portion of cannula 36. The flared guide 40, which is slidable over the exterior of the cannula 36, guides the distal end of the elongated cannula 36 into the proximal end 41 of a tubular cutting member 42 of biopsy device 43 which is described in greater detail in copending application Ser. No. 10/911,206, filed on Aug. 3, 2004 which is incorporated herein. As indicated in FIG. 7, fibrous marker 10 is disposed within the inner lumen 39 of the elongated cannula 36. A plunger 45 is slidably disposed within the inner lumen 39 of the elongated cannula 36. The plunger 44 is provided with a head 45 to facilitate pressing the plunger into the inner lumen 39 by finger pressure against the head. The distal end 46 of the plunger 44 presses against the fibrous marker 10 within the inner lumen 39 to move the marker within the lumen and to discharge the marker from the discharge opening 47 in the distal end 38 of the delivery cannula 36. Other markers (not shown) which may be disposed within the inner lumen 39 will likewise be discharged through the discharge opening 48. FIG. 8B illustrates the location of the hub 44 and flared guide 40 when the marker is discharged.

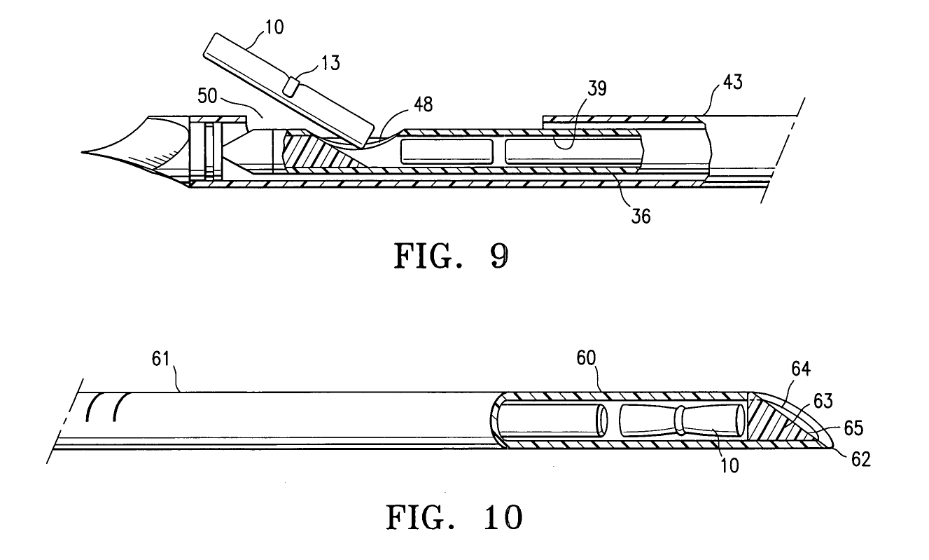

As shown in FIG. 9, the discharge opening 48 of the cannula 36 is aligned with the tissue accessing opening 50 of the biopsy device 43 to allow the fibrous marker 10 to be discharged into a body cavity (not shown). The proximal end 41 of the tissue cutter 42, the flared guide 40 and/or the distal end 38 (none shown in FIG. 9) may be provided with mating guide elements which orient the marker delivery system 35 so that discharge opening 48 of the delivery cannula 36 is properly aligned with the tissue accessing aperture 50 on the biopsy device 43. The fibrous marker 10 is advanced through the inner lumen 39 and discharged through the opening 48 and aperture 50 when the plunger 44 is urged distally within the inner lumen 39 of the delivery cannula 36. The delivery of the marker 10 to the target site after the tissue specimen has been removed, while the distal end of the biopsy device is still at the biopsy site, ensures that the marker is properly position at the site.

The fibrous marker 10 is preferably a rolled or folded piece of fibrous mat formed of a bioresorbable synthetic polymeric material, preferably PGA which has been compressed and impregnated with a binding agent such as polyethylene glycol and freeze dried in the compressed condition. Alternatively, the fibrous mat forming the fibrous body 11 may be first compressed, rolled or otherwise shaped and then impregnated with binding agent and dried. The fibrous material may be rolled up by itself or wrapped about a core. The fibrous marker 10 is generally about 0.5 mm to about 12 mm, preferably about 1 to about 8 mm in maximum transverse dimension and about 5 to about 30 mm, preferably about 10 to about 25 mm in length. Upon contact with a body fluid or other water based fluid, the length of the fibrous marker remains about the same but the wrapped structure unfolds due to the dissolution of the binding agent to a width of about 5 to about 25 mm, usually about 10 to about 20 mm. While the radiopaque marker ring 12 clamped about a center portion of the wrapped fibrous marker 10, the fibrous marker unrolls or unfolds or otherwise expands when exposed to body fluids due to the dissolution of the binding agent which holds the marker in a compressed condition. However, even though secured to the fibrous marker 10, the radiopaque marker ring 13 need not be surround the central portion of the marker as shown in the drawings, nor does it need to restrict the expansion of the fibrous marker as shown.

The manufacture of fibrous marker 10 preferably starts with a fibrous mat of PGA with a bulk density of about 40 mg/mm and having a thickness of about 0.04 to about 0.375 inch (1-9.3 mm), preferably about 0.6 to about 0.8 inch (15.4-20.3 mm) thick. The mat is rolled, impregnated with a 30% (Wt.) polyethylene glycol in a 70% isopropyl alcohol solution and then compressed. The compressed and rolled mat is then freeze dried to a diameter of about 0.065 inch (1.65 mm). Elevated temperatures may be employed to dry the fibrous material. A radiographically detectable, non-magnetic marker ring 12 may be formed of wire about 0.005 to about 0.01 inch, (0.13-0.25 mm) may then be crimped about or embedded in a central portion (or other desired portion) of the rolled and compressed fibrous body to form the fibrous marker 10. The fibrous marker 10 is then ready for deployment. Suitable fibrous material is a felt mat sold as SCAFTEX by Biomedical Structures in Slatersville, R.I.

FIG. 10 illustrates the distal portion 60 of cannula 61 of an alternative delivery system that is essentially the same as that shown in FIGS. 3A-3C except that the distal tip 62 of cannula 61 is configured in a needle-like shape. The delivery system cannula 61 may be used in conjunction with a guide cannula (not shown) or the cannula 61 can be inserted directly through tissue to reach the target site without the need for a guide cannula. The releasable plug 63 is secured in the discharge opening 64 as in the previously discussed embodiment. The exposed face 65 of the plug 63 is preferably flush with the discharge opening 64 of the distal tip 62.

Insertion of marker delivery devices embodying features of the invention may be performed with or without the aid of an imaging device, such as an ultrasound imaging device, an X-ray imaging device, a MRI device, or other imaging device. Alternatively, or additionally, insertion may be visually guided, or may be guided by palpation or by other means.

The size and composition of the strands of the fibrous body 11 and the bulk density are selected so that the fibrous marker is imageable in vivo by USI for at least 3 weeks, preferably about 4 to about 12 weeks for an effective lifespan. However, the fibrous body 11 of marker 10 should not be detectable by ultrasound after about one year, preferably not after about six months, so as to avoid interfering with subsequent site examination. The radiopaque and MRI detectable marker ring or element generally will have much longer lifespan, e.g. over a year.

While particular forms of the invention have been illustrated and described herein in the context of a breast biopsy site, it will be apparent that the device and methods having features of the invention may find use in a variety of locations and in a variety of applications, in addition to the human breast. Moreover, various modifications can be made without departing from the spirit and scope of the invention. Accordingly, it is not intended that the invention be limited to the specific embodiments illustrated. It is therefore intended that this invention to be defined by the scope of the appended claims as broadly as the prior art will permit, and in view of the specification if need be. Moreover, those skilled in the art will recognize that features shown in one embodiment may be utilized in other embodiments. Terms such as “element”, “member”, “device”, “section”, “portion”, “step”, “means” and words of similar import when used in the following claims shall not be construed as invoking the provisions of 35 U.S.C. §112(6) unless the following claims expressly use the term “means” followed by a particular function without specific structure or expressly use the term “step” followed by a particular function without specific action. All patents and patent applications referred to above are hereby incorporated by reference in their entirety.

1. A remotely imageable marker comprising a shaped fibrous body which has
a. strands formed of bioabsorbable synthetic polymeric material,
b. a bulk density of at least 10 mg/cc and
c. an in-vivo life-span of at least three weeks.

2. The marker of claim 1 wherein the fibrous body has a radiopaque element secured thereto.

3. The marker of claim 2 wherein the radiopaque element is secured to a central portion of the fibrous body.

4. The marker of claim 1 wherein the radiopaque element is non-magnetic.

5. The marker of claim 4 wherein the radiopaque element is formed of a metal selected from the group consisting of titanium, platinum, gold, iridium, tantalum, tungsten, silver, rhodium and non-magnetic stainless steel.

6. The marker of claim 1 wherein the fibrous body is a felt mat.

7. The marker of claim 1 wherein the fibrous body is a woven mat.

8. The marker of claim 1 wherein the fibrous body is a rolled fibrous mat.

9. The marker of claim 1 wherein the fibrous body is a folded fibrous mat.

10. The marker of claim 1 wherein the bioabsorbable synthetic polymeric material comprises polyglycolic acid.

11. The marker of claim 1 wherein the fibrous body is predominantly strands formed of polyglycolic acid.

12. The marker of claim 1 wherein the fibrous body is formed of at least 50% by weight of strands of polyglycolic acid.

13. The marker of claim 1 wherein the fibrous body is formed of about 85% to about 100% by weight of strands of polyglycolic acid.

14. The marker of claim 10 wherein the polyglycolic acid has a molecular weight of about 15 to about 100 kD.

15. The marker of claim 1 wherein the fibrous body has at least one bioactive component incorporated therein.

16. The marker of claim 15 wherein the bioactive component is a therapeutic or diagnostic agent.

17. The marker of claim 16 wherein the therapeutic or diagnostic agent is selected from the group consisting of hemostatic agents, anesthetic agents, coloring agents, chemotherapeutic agents, radioactive agents, antibiotic agents, antiviral agents and antifungal agents.

18. The marker of claim 1 wherein the fibrous body is formed into a shape and at least in part held in the shape by a binding agent.

19. The marker of claim 18 wherein the binding agent is a water soluble polymer.

20. The marker of claim 19 wherein the binding agent is a water soluble polymer selected from the group consisting of polyvinyl alcohol, polyethylene glycol and polyvinyl pyrollidone.

21. The marker of claim 7 wherein the fibrous mat is about 0.25 to about 1.5 inch long, about 0.25 to about 1.0 wide and about 0.02 to about 0.4 inch thick.

22. The marker of claim 7 wherein the rolled fibrous mat is about 0.5 to about 12 mm thick.

23. The marker of claim 7 wherein the fibrous mat is about 1 to about 8 mm thick.

24. The marker of claim 1 wherein the fibrous body is about 5 to about 30 mm in length.

25. The marker of claim 1 wherein the fibrous body is about 10 to about 25 mm in length.

26. The marker of claim 3 wherein the radiopaque element at least in part surrounds a central portion of the fibrous body.

27. The marker of claim 1 wherein the fibours body has a bulk density of about 30 to about 100 mm/cc.

28. The marker of claim 1 wherein the fibrous body is compressed before shaping.

29. The marker of claim 1 wherein the fibrous body is compressed after shaping.

30. The marker of claim 1 wherein the fibrous body is bound after being compressed and shaped.

31. A biopsy site marker delivery system, comprising:
a. an elongated tubular shaft which has a distal end, a proximal end, an inner lumen extending between the proximal and distal ends and a discharge opening in a distal shaft section;
b. at least one site marker slidably disposed in the inner lumen of the shaft, comprising a shaped fibrous body which has a bulk density of at least 10 mg/cc, which is formed of strands of bioabsorbable, synthetic polymeric material and which has an in-vivo life-span of at least three weeks; and
c. a plunger element which is slidably disposed in part within the inner lumen of the tubular shaft proximal to the site marker and which is configured to urge the site marker out the discharge opening in the distal shaft section of the elongated tubular shaft.

32. The biopsy site marker delivery system of claim 31 wherein the elongated shaft has a flared attachment to the distal tip or a flared distal tip to facilitate the engagement a proximal end of a tubular cutting element of a biopsy element.

33. The biopsy site marker delivery system of claim 31 wherein the distal end of the elongated shaft is blocked by a releasable plug.

34. The biopsy site marker delivery system of claim 31 wherein the fibrous body has a radiopaque element secured thereto.

35. The biopsy site marker delivery system of claim 34 wherein the radiopaque element is secured to a central portion of the fibrous body.

36. The biopsy site marker delivery system of claim 35 wherein the radiopaque element at least in part encircles the central portion of the fibrous body.

37. The biopsy site marker delivery system of claim 31 wherein the synthetic polymer strands consists essentially of strands of polyglycolic acid.

38. The biopsy site marker delivery system of claim 37 wherein the synthetic polymer strands contain at least about 50% by weight polyglycolic acid.

39. The biopsy site marker delivery system of claim 37 wherein the synthetic polymer strands contain at least about 85% by weight polyglycolic acid.

40. The biopsy site marker delivery system of claim 31 wherein the fibrous body contains at least 50% by weight of strands of polyglycolic acid.

41. The biopsy site marker delivery system of claim 31 wherein the fibrous body contains about 85% to about 100% by weight of strands of polyglycolic acid.

42. The biopsy site marker delivery system of claim 31 wherein the fibrous body is formed of a felt mat.

43. The biopsy site marker delivery system of claim 42 wherein the fibrous body is shaped by rolling the felt mat into an elongated body.

44. The biopsy site marker delivery system of claim 42 wherein the felt matt is formed of strands consisting essentially of polyglycolic acid.

45. The biopsy site marker delivery system of claim 37 wherein the polyglycolic acid has a molecular weight of about 15 to 100 kD.

46. The biopsy site marker delivery system of claim 31 wherein the fibrous body has a bulk density of about 30 to about 100 mg/cc.

47. A method for delivering at least one marker body to an intracorporeal target tissue site within a patient, comprising:
a. providing a marker delivery device for delivery of a marker body which includes:
i. an elongated shaft which has an inner lumen, a discharge opening in a distal portion of the elongated shaft;
ii. at least one fibrous marker body which has a bulk density of at least 10 mg/cc, which is formed of strands of bioabsorbable, synthetic polymeric material, which is slidably disposed within the inner lumen of the elongated shaft and which has an in-vivo life-span of at least three weeks; and
iii. a plunger element which is slidably disposed within the inner lumen of the marker delivery device proximal to the fibrous marker disposed therein and which is configured to urge the fibrous marker out the discharge opening in the distal portion of the elongated shaft;
b. advancing the marker delivery device within the patient until the distal end of the marker delivery device is disposed at the target tissue site and the discharge opening of the marker delivery device is aligned for marker deployment; and
c. pressing the plunger element of the marker delivery device to eject the fibrous marker body through the discharge opening in the marker delivery device and into the target site.

48. A method for delivering at least one marker body to an intracorporeal target tissue site within a patient, comprising:
a. the step of providing a marker delivery device for delivery of a marker body which includes:
i. an elongated shaft which has an inner lumen, a discharge opening in a distal portion of the elongated shaft,
ii. at least one fibrous marker body which has a bulk density of at least 10 mg/cc, which is formed of strands of bioabsorbable, synthetic polymeric material, which is slidably disposed within the inner lumen of the elongated shaft and which has an in-vivo life-span of at least three weeks, and
iii. a plunger element which is slidably disposed within the inner lumen of the marker delivery device proximal to the fibrous marker disposed therein and which is configured to urge the fibrous marker out the discharge opening in the distal portion of the elongated shaft;
b. the step of advancing the marker delivery device within the patient until the distal end of the marker delivery device is disposed at the target tissue site and the discharge opening of the marker delivery device is aligned for marker deployment; and
c. the step of pressing the plunger element of the marker delivery device to eject the fibrous marker body through the discharge opening in the marker delivery device and into the target site.
